(19) United States
(12) Patent Application Publication
(10) Pub. No.: US 2011/0208100 A1
(43) Pub. Date: Aug. 25, 2011

(54) Method and Apparatus for Treating Carpal Tunnel Syndrome

(76) Inventors: Donald R. Eck, Hesperia, MI (US); Jerome V. Eck, Sterling Heights, MI (US)

(21) Appl. No.: 12/773,337

(22) Filed: May 4, 2010

Related U.S. Application Data
(60) Provisional application No. 61/306,152, filed on Feb. 19, 2010.

(51) Int. Cl. A61F 5/00

(52) U.S. Cl. 602/21

(57) ABSTRACT

A method and apparatus for treating carpal tunnel syndrome with the use of an orthotic. The orthotic is applied to the patient's hand. The use of the orthotic by the patient is monitored automatically and data is produced as to the amount and time of usage. This data is then compared with a protocol to determine if the patient usage is in compliance with the protocol.

### CROSS-REFERENCE TO RELATED APPLICATIONS

This Application claims the benefit of U.S. Provisional Application No. 61/306,152 filed Feb. 19, 2010 and is incorporated by reference in its entirety.

### STATEMENT REGARDING FEDERALLY SPONSORED RESEARCH OR DEVELOPMENT

Not Applicable

### THE NAMES OF THE PARTIES TO JOINT RESEARCH

Not Applicable

### BACKGROUND OF THE INVENTION

1. Field of the Invention

The invention is related to a method and apparatus for treating carpal tunnel syndrome in a human patient. The method and apparatus are used to treat carpal tunnel by reducing or alleviating median nerve compression through stretching the muscles and their aponeurotic insertions. An orthotic in the form of an external brace is applied to the band and wrist.

2. Historical Background

Carpal tunnel syndrome is a pathological condition of the upper extremity and of the hand and fingers caused by compression of the median nerve resulting in pain, numbness, paresthesia, and weakness of the affected part. Carpal tunnel syndrome has come into prominence because of hand intensive activities of workers in industry, its work related morbidity, and employee loss of work time.

Moderate and severe cases of carpal tunnel syndrome have succumbed to surgical intervention for relief of the symptoms; sometimes with good results and sometimes with less than satisfactory results.

It has been found that most of the compression to the median nerve was distal to the carpal tunnel ligament and that the other structures contained within the ligament were not involved in a pathological process. After having performed many carpal tunnel operations and decompressing the affected median nerve, it was concluded that the carpal tunnel ligament and its contents were not the etiological agents of this disease or condition, but that the thenar and hypothenar muscles and their aponeurotic insertion were etiological factors to be considered.

Carpal tunnel syndrome results in the second highest number of workdays lost among all work related injuries (National Center for Health Statistics). Millions of Americans experience carpal tunnel syndrome symptoms. The use of computer keyboards is driving up carpal tunnel syndrome ten percent per year. Carpal tunnel syndrome related costs are continuing to increase.

There are over 500,000 carpal tunnel surgeries per year in the U.S.A. The surgery is not always effective and carpal tunnel symptoms persist after one third of the carpal tunnel surgery cases. If symptoms do return, surgeons require change in repetitive stress work patterns, change of job or career change before performing a second surgery.

Most tendons insert into the bone proximally and distally giving motion to joints in the body. Unlike most tendons, the thenar and hypothenar group muscles insert into a common tendon proximally, tendinous-to-tendinous insertion, this is called an aponeurosis. Skeletal muscle tissue has the properties of irritability, contractibility, and extensibility. When muscle is intensively used, the muscle components enlarge and muscle mass is increased. This increase in muscle mass is called hypertrophy. Hypertrophy causes an increase in muscle tone and results in an increase in the myotatic reflex of the thenar and hypothenar muscles groups. What follows is an increased tension in the aponeurotic tendon structure causing compression of the sub adjacent median nerve, resulting in CTS symptoms.

3. Description of the Related Art

Orthotics have been developed to relieve the pressure caused by the aponeurotic tightness over the median nerve caused by compression to the nerve distal to the carpal tunnel ligament. U.S. Pat. No. 5,746,707 issued May 5, 1998 for Carpel (sic) Tunnel Syndrome External Brace, Donald R. Eck shows a wrist and two digit restraining devices for the treatment of carpal tunnel syndrome. This design decreases the necessity for surgical intervention of the patient.

The brace performs three functions to accomplish the desired results:
1. a slight angular extension of the affected wrist;
2. a position of stretch for the thumb and fifth digit; and
3. the allowance of free motion of the second, third and fourth digits of the involved extremity.

The brace includes a main support portion which extends under the wrist portion of the arm and the heel and the palm of the hand of the patient's extremity and outwardly extending support portions for the thumb and fifth digit of the hand so that the thumb and fifth digit are in a fully abducted position. A wrist strap adjacent to the inner end of the brace clamps the brace onto the wrist and secondary straps are used to bind the thumb and fifth digit onto the supports for these digits. Over a period of time, use of this brace results in a marked reduction of night pain, numbness and paresthesia of the affected hand.

U.S. Pat. No. 5,746,707 is incorporated by reference in its entirety herein.

4. Problem

There is no known system to determine the success or failure of the orthotic other than the veracity of the patient. For example the patient is fitted for an orthotic and is told to wear it so many hours a day. The patient returns in a week and says she has been wearing the orthotic for the prescribed period of time and tells the physician the pain is worse.

One of two things exist. One, the patient is not stating the time of usage accurately. This can be intentional or unintentional. Two, the patient is stating the usage accurately.

There is no known orthotic now in existence for carpal tunnel that can indicate if a patient has followed the protocol established and recommended by the physician. Currently the physician must accept the statement of usage by the patient at face value. Relying only on the patient's statement of use is not acceptable in some situations. One of those situations is where the patient has a monetary incentive not to be truthful. If the patient intentionally misstates that the orthotic has been worn as directed and there is still pain, and other symptoms persist the patient can file a claim for workers compensation and other claims for disability to avoid work.

There is a need to confirm a patient's statement of use. Financial compensation for patient claims can be granted or denied based upon the patient's usage of the orthotic. Accurate information by a well intentioned patient will assist the physician or medical advisor in a course of treatment for the patient such as recommending surgery or more aggressive use of the orthotic.

### BRIEF SUMMARY OF THE INVENTION

The invention reduces or alleviates median nerve compression through stretching the muscles and their aponeurotic insertions.

The patient consults with a physician or a medical clinician to determine the patient's need. The patient will report symptoms occurring as a result of carpal tunnel syndrome. If the need for an orthotic exists the patient is presented with an orthotic designed to reduce the nerve compression and is given instructions on when, where and how long to use the orthotic.

The invention presents a solution to the problem of determining the patient's compliance with the instructions to the patient on use of the orthotic that is independent of the patient's statement of usage or best recollection of the patient. The compliance is determined by a system and structure.

We provide a method for treating carpal tunnel syndrome in a patient by providing an orthotic that is custom configured to a patient's hand and wrist. The orthotic is applied to the patient. Usage is monitored automatically and produces data from the monitoring in which the data gives the amount of the usage to assist one to determine further course of treatment of the patient's carpal tunnel syndrome.

The amount of usage data is compared with a protocol. The data is coupled to a computer into which the data is downloaded and compared with the protocol. The monitoring includes the use of a monitoring device that produces the data. The data is obtained by interfacing between the monitoring device and the patient. The monitoring device is within the orthotic. The monitoring device senses temperature over time through an interface between the monitoring device and the patient and produces data giving the temperature over time.

A patient identification is associated with the monitoring device and the data produced from it.

We provide a method for determining compliance by a patient of an amount of usage by a patient of an orthotic for treating carpal tunnel syndrome with recommended usage to the patient by a medical practitioner. An orthotic configured for treating carpal tunnel syndrome is provided. The patient's amount of usage of the orthotic is monitored automatically over a period of time. Data is produced from the monitoring in which the data gives the amount of the usage by the patient over the period of time to assist the medical practitioner to determine whether the patient has complied with an amount of recommended usage to the patient by the medical practitioner.

We provide an apparatus for treating carpal tunnel syndrome comprising an orthotic device configured in a shape for treating carpal tunnel in a patient. A monitoring device is attached to the orthotic and collects data showing temperature over time at an interface between the patient and the monitoring device when the patient is wearing the orthotic. A computer is coupled to the monitoring device to receive the data from the monitoring device and compares the data with a protocol.

We provide an apparatus for determining compliance of a patient of a recommended usage of an orthotic for treating carpal tunnel syndrome. An orthotic is configured in a shape for treating carpal tunnel in a patient. A monitoring device is inserted within the orthotic. The monitoring device collects data showing an amount of usage of the orthotic by the patient over time at an interface between the patient and the monitoring device when the patient is wearing the orthotic. A computer is coupled to the monitoring device to receive the data from the monitoring device in which the computer compares the data that shows the amount of usage by the patient with a protocol of usage recommended to the patient by a medical practitioner.

### BRIEF DESCRIPTION OF THE DRAWINGS

FIG. 1 is a perspective view of the orthotic showing a dorsal view.

FIG. 2 is a perspective view of the orthotic showing a palmar view.

FIG. 3 is a perspective view of the orthotic with cloth covering.

FIG. 4 is a perspective view of the orthotic with cloth covering showing the orthic attached to a hand and wrist.

FIG. 5 is a perspective view of the portion of the orthotic that is flipped upside down and showing a data logger and a data reader.

### DETAILED DESCRIPTION OF THE INVENTION

1. Definitions of Terms Used

The following definitions do not limit the terms but are an indication of what can be included within the meaning of the terms.

Treating carpal tunnel syndrome—using therapy to reduce the symptoms of carpal tunnel

Carpal tunnel syndrome—a repetitive strain disorder or cumulative trauma disorder because of the repetition forces that act as a catalyst for this condition. It has been variously classified as a disease, an illness, a disorder, a syndrome and/or an injury.

Orthotic—a device that can act as a brace to keep a body part in a certain position

Applying the orthotic to the patient—the patient's hand and/or wrist is joined with the orthotic by suitable means to keep a patient's hand, finger and wrist in a fixed position

Monitoring—keeping a continuous surveillance over a period of time

Automatically—a process or a device working by itself in which at least some portion has no direct human intervention

Usage of the orthotic—the patient has the orthotic applied to the patient's hand over a period of time or at certain times over a period of time

Data—facts in electronic form collected for reference or analysis

Protocol—procedure or standard for carrying out a course of medical treatment; or can also include instructions or a standard provided to a carpal tunnel patient to follow in use of an orthotic

Inter facing between the monitoring device and the patient—a point in proximity between the hand or wrist of the patient and the monitoring device

Medical practitioner—a physician or a person or medical advisor qualified to assist a physician in the treatment of carpal tunnel or the use of an orthotic by a patient

2. The Structure

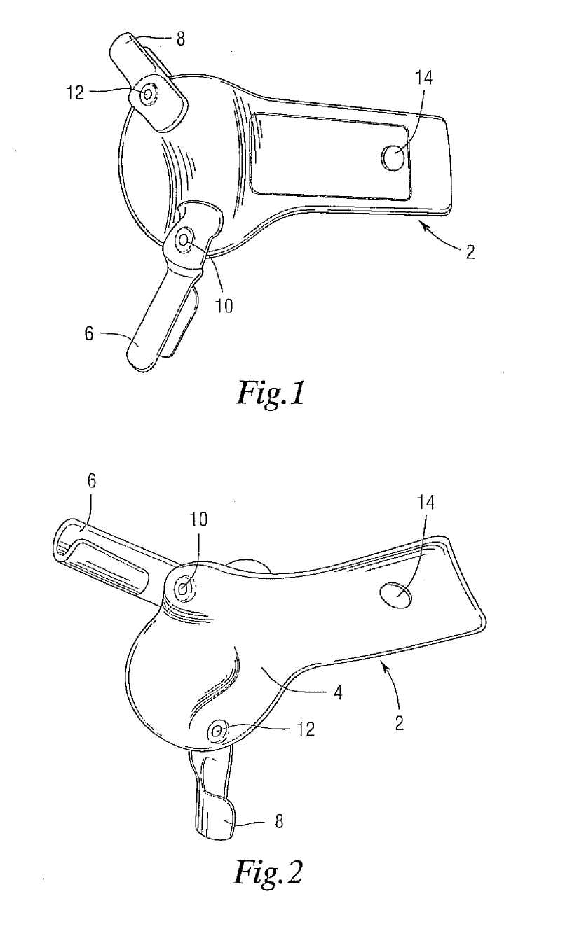

FIGS. 1-4 show the orthotic 2 that has a molded plastic hand section 4 (FIG. 2) with two molded plastic finger restrictor devices 6 and 8. The orthotic 2 is configured with the finger restrictor devices 6 and 8 which are shaped to receive fingers 24 and 26 on a hand 22 and keep the fingers 24 and 26 separated apart from each other. Restrictor devices 6 and 8 are pivotally mounted at points 10 and 12 respectively so that the restrictor devices 6 and 8 can be moved to different angles.

A monitoring device 14 is inserted into the orthotic 2 in proximity on the orthotic 2 where a wrist would interface with the monitoring device 14 as shown in FIG. 2. The monitoring device 14 records time and temperature. This is available from Maxim Integrated Products and is referred to by Maxim Integrated Products as an i Button.

The i Button is a computer chip enclosed in a 16 mm thick stainless steel can. Up to date information can travel with a person or object. The i Button uses its steel can as an electronic communication interface. The can has a data contact called the “lid” and a ground contact called the “base”. Each of these contacts is connected to a silicon chip inside. The lid is the top of the can; the base forms the sides and the bottom of the can and includes a flange to attach the i Button to another structure. The two contacts are separated by a polypropylene grommet.

Touching the i Button to the above contacts described enables one to communicate to a computer through a one wire system.

Each i Button has an unalterable address laser etched onto its chip inside the can. The address (e.g. 2700108) can be used as a key or an identifier for each i Button.

Information is transferred between the i Button and a personal computer (PC) with a monitoring contact at up to 142 KBPS.

This is done by touching the i Button to a probe (receptor) which is connected to a PC spare port such as a USB.

Software is used to interface the i Button to a computer and produce the desired information in the desired format.

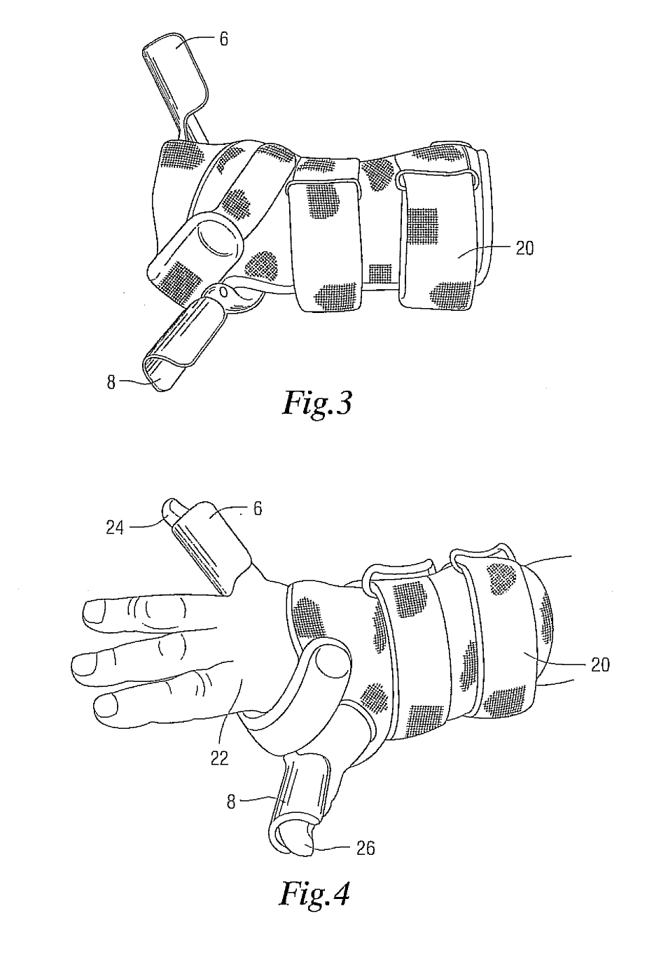

FIGS. 3-4 shows the orthotic 2 with a cloth covering 20 which holds a hand 22 with fingers 24 and 26 inserted into finger restrictor devices 6 and 8 respectively.

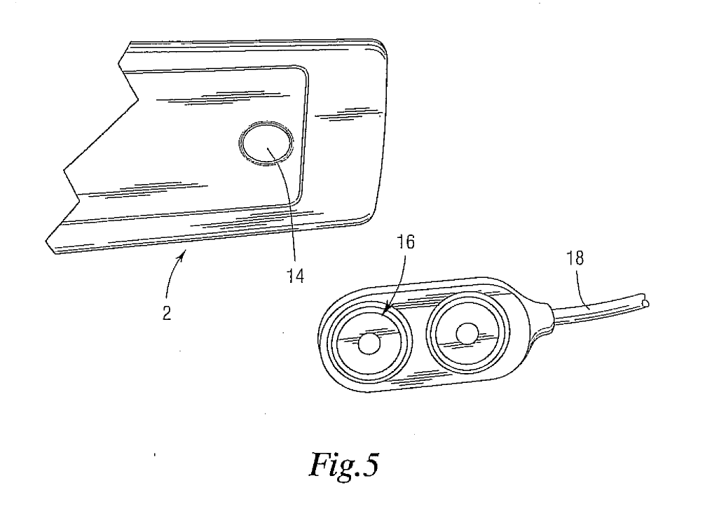

FIG. 5 shows the i Button (monitoring device 14) in the orthotic 2. A separate reader contact 16 (also known as a one wire system-probe) with a cord 18 plugs into a USB port on a PC (not shown).

3. The Method

The orthotic 2 is designed to specifically cause a stretching of the aponeurotic insertion of the conjoined tendons of thenar and hypothenar muscles and the flexor retinaculum by placing and maintaining the position of the hand 22 and wrist for a sustained period of time while the patient sleeps. The position is acute extension of the wrist and abduction of 1^{st} and 5^{th} fingers 26 and 24.

Prolonged periods of stretching result in the fatigue of the thenar and hypothenar muscles and the flexor retinaculum causing a relaxation of the conjoined tendons and subsequent decompression of the subjacent median nerve. This decompression allows for normalization of the vascular flow beneath and distal to the compression site. This decompression also allows for normalization of tissue and nutrients to the nerve, causing a relief of symptoms.

The patient is examined for carpal tunnel syndrome. A plan for therapy is created. If the use of an orthotic 2 is a part of the plan the patient gives an imprint of the hand 22 and wrist in a foam material. An orthotic 2 is constructed specifically to the anatomical configuration of the patient.

The orthotic 2 has a monitoring device 14 which records time and temperature. The temperature is obtained by interfacing between the monitoring device 14 and the patient's hand 22/or wrist. Because the patient's hand 22/wrist are adjacent/near the monitoring device 14 the temperature at the monitoring device 14 is generally higher than room temperature and moves upwardly toward body temperature which is higher than room temperature.

When the patient's hand 22 is in the orthotic 2 an elevated temperature is reached and the time that this elevated temperature is present is recorded by the monitoring device 14 with the temperature. This tells one how long the patient has been wearing the orthotic 2 which gives an objective and automatic amount of usage of the orthotic 2 by the patient. It can also give a total time and when during that total time the patient is wearing the orthotic. For example, is the patient wearing the orthotic during sleeping hours? This eliminates relying solely upon the patient's statement as to how long or the amount of usage when the orthotic 2 is used by the patient.

The time and temperature are kept as data in the monitoring device 14. The monitoring is automatic and is not manipulated by the patient.

The monitoring device 14 has a serial number. This number is then associated with a patient and becomes the identification number that is associated with the orthotic, data and patient.

This system tracks the time and temperature when the orthotic 2 is being worn by the patient. This in turn gives the data of use as to when and how much the orthotic 2 is used by the patient.

This usage can then be compared with a protocol established by a medical advisor or physician. The comparison tells the physician or medical advisor if the patient has complied with the established protocol. This usage compliance determination is totally independent of any subjective statement uttered to the physician or medical advisor by the patient.

When the patient returns to the physician or medical advisor's office the patient will give the orthotic 2 to the physician or medical advisor. The physician or medical advisor will remove the orthotic 2 from the cloth covering 20 and place the reader contact 16 (FIG. 5) so that it touches the monitoring device 14. The reader contact 16 is connected to a PC USB through a cord 18. Data representing temperature over time, which has been collected by the monitoring device 14 when the orthotic was worn by the patient, is transferred to a PC. When the data is uploaded into the PC it can be compared with a protocol established by the physician or medical advisor. This comparison will tell the physician or medical advisor if the patient's usage was in compliance with the protocol established by the physician or medical advisor.

If the patient's usage is found to be in compliance with the established protocol and the symptoms of the carpal tunnel syndrome persist without improvement then the physician or medical advisor will decide what future treatment should be considered. This system assists the physician or medical advisor on the course of future treatment.

If on the other hand the patient is found not to be compliant with the protocol then the medical advisor knows that whether or not the orthotic would be effective cannot be determined because the patient is not using the orthotic as instructed by the physician or medical advisor. Software is provided for: uploading the collected data from the monitoring device 14 to the PC; uploading the protocol information to the PC; and comparing the collected data with the protocol to determine if the patient was compliant in the usage recommended by the physician or medical advisor.

Examples of Protocol

A. Simple Protocol

The patient is instructed to wear the orthotic by the medical advisor 6 to 8 hours per day while sleeping. This is to be continued for 6 weeks.

B. More Involved Protocol
1) Fitting the orthotic to the patient and commissioning (starting) the chip and recording patient's subjective complaints:

a) Night pain—on a scale from 1 to 10, 10 being worst.

b) Pain intensity—on a scale from 1 to 10, 10 being worst

c) Numbness—based on a scale of 1 to 5, 5 being worst

d) Paresthesia—based on a scale of 1 to 5, 5 being worst

e) Tactile difficulty—based on a scale of 1 to 5, 5 being worst

The patient to wear orthotic 6 to 8 hours per day while sleeping, for a duration of 6 weeks.
2) One week post fitting and application.
   The physician or medical advisor records chip activity again using USB port which is transmitted to a secure database center as above. Patient subjective symptoms are again recorded and placed in database record.
3) Second week post fitting and application.
   The physician or medical advisor records chip activity using USB port which is transmitted to a secure database center as above with the subjective symptoms.
4) Sixth week post fitting and application.
   Final recording of chip and patient's subjective symptoms and transmitting to secure database.
   Patient can now use orthotic on an “as needed basis” to control carpal tunnel symptoms.
5) Suggested Electromyogram 6 months following initial treatment to be compared to pre-treatment Electromyogram if available.

### Advantages

1) No loss of work
2) No change of job or career
3) Life-time treatment system
4) Marked reduction of treatment cost and capitation of costs

Various changes can be made to the above method and apparatus without departing from the scope of the invention as defined in the claims below. It is intended that all matter contained in the above description as shown in the accompanying drawings shall be interpreted as illustrative and not as a limitation.

We claim:

1. Method for treating carpal tunnel syndrome in a patient comprising:
a) providing an orthotic configured for treating carpal tunnel;
b) applying the orthotic to the patient;
c) monitoring automatically usage of the orthotic by the patient; and
d) producing data from the monitoring in which the data gives the amount of the usage to assist one to determine further course of treatment of the patient's carpal tunnel syndrome.

2. Method for treating carpal tunnel syndrome in a patient as recited in claim 1 including comparing the amount of usage data with a protocol.

3. Method for treating carpal tunnel syndrome in a patient as recited in claim 1 where the data is coupled to a computer into which the data is downloaded and the data is compared with a protocol.

4. Method for treating carpal tunnel syndrome in a patient as recited in claim 1 where the monitoring includes a monitoring device and the data that is produced comes from the monitoring device.

5. Method for treating carpal tunnel syndrome in a patient as recited in claim 4 where the data is obtained by interfacing between the monitoring device and the patient.

6. Method for treating carpal tunnel syndrome in a patient as recited in claim 4 where the monitoring device is within the orthotic.

7. Method for treating carpal tunnel syndrome in a patient as recited in claim 4 where the monitoring device senses temperature over time at an interface between the monitoring device and the patient and produces data giving the temperature over time.

8. Method for treating carpal tunnel syndrome in a patient as recited in claim 4 including a patient identification associated with the monitoring device and the data that is produced.

9. Method for determining compliance by a patient of an amount of usage by a patient of an orthotic for treating carpal tunnel syndrome with a recommended usage to the patient by a medical practitioner comprising:
a) providing an orthotic configured for treating carpal tunnel;
b) monitoring automatically the patient's amount of usage of the orthotic over a period of time; and
c) producing data from the monitoring in which the data gives the amount of the usage by the patient over the period of time to assist the medical practitioner to determine whether the patient has complied with an amount of recommended usage to the patient by the medical practitioner.

10. An apparatus for determining compliance of a patient of a recommended usage of an orthotic for treating carpal tunnel syndrome comprising:
a) an orthotic device configured in a shape for treating carpal tunnel in a patient;
b) a monitoring device within the orthotic, the monitoring device collects data showing an amount of usage of the orthotic by the patient over time at an interface between the patient and the monitoring device when the patient is wearing the orthotic; and
c) a computer coupled to the monitoring device to receive the data from the monitoring device in which the computer compares the data that shows the amount of usage by the patient with a protocol of usage recommended to the patient by a medical practitioner.
